(19) United States
(12) Patent Application Publication
(10) Pub. No.: US 2018/0132717 A1
(43) Pub. Date: May 17, 2018

(54) ECG Electrode and Method for Detecting an ECG Measurement Signal

(71) Applicant: Ulrich Batzer, Buckenhof (DE)

(72) Inventor: Ulrich Batzer, Buckenhof (DE)

(21) Appl. No.: 15/811,413

(22) Filed: Nov. 13, 2017

(30) Foreign Application Priority Data
Nov. 14, 2016 (DE) 102016222305.4

(51) Int. Cl. A61B 5/00; A61B 5/0408

(52) U.S. Cl. CPC A61B 5/0006; A61B 5/6823; A61B 5/0408

(57) ABSTRACT

An electrocardiography (ECG) electrode includes a contact element carrier for encompassing a first extremity of a patient, and a first skin contact element arranged on the contact element carrier with a first connection element for detecting a first potential on the first extremity. The ECG electrode also includes at least one second skin contact element arranged on the contact element carrier with a second connection element for detecting a second potential different from the first on the first extremity.

This application claims the benefit of DE 10 2016 222 305.4, filed on Nov. 14, 2016, which is hereby incorporated by reference in its entirety.

### BACKGROUND

The present embodiments relate to an electrocardiography (ECG) electrode.

In modern medicine, ECG measuring systems are used to measure heart signals of a patient or subject (hereinafter jointly referred to as “patient” for the sake of brevity), for example, of a human or animal. Usually, electric potentials for measuring or deriving the heart signals depending on the application are detected by an ECG measuring device with ECG electrodes connected thereto at at least two (e.g., at least three) contact points on the skin of the patient. The minimum number of contact points is based on the fact that in the minimum configuration, two contact points are used for differential measurement, and a further, third contact point is used for equipotential bonding. The ECG electrode for equipotential bonding is also referred to as a neutral electrode or “Right Leg Drive” (RLD). However, the number of contact points may be up to ten or more to enable analysis of the potential of the heart from various angles and/or to achieve better electromagnetic interference suppression.

The number of ECG electrodes used depends, among other things, on the purpose for which the ECG measurement signal is to be recorded, whether, for example, the ECG measurement signal is to be used for a more specific qualitative evaluation of cardiac function, or whether, for example, only one trigger point is used to initiate an imaging measurement. For example, for an imaging measurement of the heart with the medical imaging devices currently available, such as computer tomography systems, magnetic resonance tomography systems, angiography systems, and the like, it is useful to trigger data acquisition times with the aid of the use of an ECG measurement signal such that the data may be recorded in a particular phase of the cardiac activity (e.g., within a resting phase).

The ECG electrodes may be configured and arranged such that the ECG electrodes affect the patient as little as possible. At the same time, however, a good signal quality may also be provided. For a good measurement, the most important parameters are the positioning of the ECG electrodes such that a sufficient amplitude of the ECG measurement signal is generated, that there is the least contact resistance possible between the ECG electrode and the skin, that the fixation of the ECG electrodes on the patient is as free of play as possible, and that a shield against electromagnetic interference coupling is provided. In a clinical setting (e.g., in an application on a patient in a medical imaging device), there are further constraints that are partially at odds with this. First, this pertains to as little negative impact on the patient as possible by the ECG electrodes, and second, this pertains to easy application or simple attachment of the ECG electrodes to the patient (e.g., also including physically disabled or unconscious patients). In addition, it should be provided that there is no impairment of the signal quality of the imaging signal in the imaging devices, for example, as a result of metal parts such as cables or ECG electrodes inside the ray path of a computer tomography system or inside the patient tunnel of a magnetic resonance tomography system. For certain diagnostic applications, attention should also be paid to conformity with normative requirements (e.g., the exact positioning of the ECG electrodes).

To enable high quality measurements of an ECG, adhesive or suction electrodes that are positioned on the chest surface of the patient may be used. With these electrodes, a chest derivative is then possible as a result of placing a plurality of electrodes around the rib cage. The quality is almost ideal, but the patient is to undress for this, and the electrodes are time-consuming to apply and are to be removed again after measurement. If, for example, such a multiplicity of ECG derivatives is not necessary in order to obtain a simple trigger signal, but only the so-called Einthoven derivatives I to III between the right and left arm and the left foot are to be measured, clamping electrodes are also customary in the clinical setting. In this case, these are flattened, annular clamps on each of which a skin contact element is arranged at a point on the inside and which, when clamping the clamping electrodes on the arm or leg (e.g., on the wrist or just above the wrist or on the ankle or just above the ankle), is pressed against the skin of the patient. However, the patient is restricted in freedom of movement by the three clamping electrodes. In addition, in a clinical setting, use on the leg is sometimes time-consuming for in-patients as the in-patients are often wearing compression stockings.

In the fitness and leisure sector, ECG electrodes on chest straps are known. However, ECG electrodes on chest straps have the disadvantage that the electrodes are very time-consuming to attach to physically disabled patients and in imaging devices impair imaging in the proximity of the heart as a result of high metal content. In the fitness sector and in the field of telemedicine, there are special watches with a contact on the top and bottom of the housing, respectively. The lower contact is always in contact with the wrist of the user, usually on the left arm, and the upper contact is to be touched by the user with the right hand for measurement. Thus, an Einthoven I measurement arises from the right arm to the left arm. However, this method requires the active participation of the patient. The patient is to be in contact with the contact surface of the watch, as far as possible with constant pressure from the right hand. In the event of movement or varying pressure, the electrical properties of the contact change and signal interference occurs. For application in a clinical setting (e.g., in order to obtain trigger signals for imaging), such watches or the like are therefore unsuitable.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an ECG electrode and a method for detecting an ECG measurement signal, by which the least possible negative impact on the patient, the least possible interference in an imaging measurement, and adequate signal quality are provided, are provided.

The ECG electrode according to one or more of the present embodiments has a contact element carrier for encompassing a first extremity of a patient. This encompassing may take place essentially positively (e.g., in a manner such that, at least in sections, the extremity is acted upon as closely as possible by the contact element carrier). Reference to examples of this will be made later. A first skin contact element is arranged on the contact element carrier with a connection element assigned to this skin contact element for a first input channel or measuring channel of an ECG measuring device. This first connection element may, for example, be a plug contact into which a cable of the ECG measuring device may be inserted in a conventional manner, but also, where appropriate, a fixed conductor section or a cable for an ECG measuring device integrated into or on the ECG electrode, as will also be explained later. This first skin contact element is used to detect, measure, or tap a first potential (e.g., an electrical signal concerning the potential change on the first extremity).

Unlike a conventional clamping electrode, on the contact element carrier according to one or more of the present embodiments, there is also at least one second skin contact element with a second connection element assigned to this second skin contact element for a second input channel of the ECG measuring device. This second connection element may also be, for example, a plug contact, a conductor section, or the like. This second skin contact element is used to detect a second potential different than the first potential on the first extremity. Each of the skin contact elements is configured to establish an electrical contact with the skin when the electrode is attached to the skin. To be able to detect two different potentials, the skin contact elements and the associated connection elements for the two input channels are therefore electrically isolated from each other in or on the ECG electrode. In contrast to a previously customary ECG electrode (e.g., an aforementioned conventional clamping electrode), it is possible to detect two potentials on the same extremity with only one ECG electrode.

This ECG electrode according to one or more of the present embodiments may be part of an ECG electrode set that has a number of ECG electrodes. At least one of the ECG electrodes is an ECG electrode. In one embodiment, two ECG electrodes may be used for essentially positive connection to a first extremity and to a second extremity of the patient. The second ECG electrode is used to detect a third potential on the second extremity. In other words, only two ECG electrodes are then necessary for the arms, for example, and an application on the leg is unnecessary.

As will be explained later, the first skin contact element or the second skin contact element is used to detect a reference potential (e.g., as a replacement for the neutral electrode (Right Leg Drive)).

An ECG measuring system according to one or more of the present embodiments includes such an ECG electrode set with at least one ECG electrode.

The method according to one or more of the present embodiments for detecting an ECG measurement signal or an ECG derivative (e.g., an Einthoven derivative I to III) tapping, on a first extremity (e.g., an arm of a patient) a first potential and a second potential different from the first potential off by an ECG electrode. In one embodiment, at least a third potential is detected on another part of the body of the patient.

Further embodiments and developments will emerge from the following description, where a category may also be developed analogous to the features and exemplary embodiments of another category. For example, individual features of different exemplary embodiments or versions of new exemplary embodiments or versions may also be combined.

There are various options for the realization of the contact element carrier (e.g., the part on which the skin contact elements are fastened). In one embodiment, the contact element carrier has a band or a ring element (e.g., a ring segment, a half-ring, etc.). A ring may be a slotted ring (e.g., also ring segments linked to each other via a hinge or the like, as in the case of the customary clamping electrodes described later).

The first skin contact element and the second skin contact element are arranged on an inner side of the contact element carrier pointing towards the skin of the patient in the intended use of the ECG electrode (e.g., on essentially opposite band or ring sections). If the skin contact elements abut the extremity (e.g., the wrist or ankle, the fingers, the toes, etc.) approximately diametrically opposed on opposite sides or different ring segments of the contact element carrier, this provides that the first skin contact element and the second skin contact element on the contact element carrier abut the most remote contact points possible of the extremity on the skin of the patient.

In one embodiment, the contact element carrier for encompassing a forearm or lower leg may be configured as a wrist and/or ankle clamp or as a wrist and/or ankle band, for example, in the form of a belt with a clasp for tightening. Fastening therefore takes place directly on or next to the respective joint on the forearm or lower leg.

In one embodiment, the first skin contact element and/or the second skin contact element each has a contact surface with the first extremity that is at least 0.5 cm², at least 1 cm², or at least 1.5 cm². The contact surface may be 5 cm² maximum, 3 cm² maximum, or 2.5 cm² maximum. In one embodiment, the size of the contact surface is between 1.5 cm² and 2.5 cm².

To avoid longer wiring from the ECG electrode to an ECG measuring device, in a version, the ECG electrode itself may also have an integrated ECG measuring device that, for example, is arranged in a housing on the contact element carrier.

In one embodiment (e.g., when such an integrated ECG measuring device is included), the ECG electrode has an energy storage system (e.g., an accumulator or a battery) to operate the ECG electrode (e.g., the integrated ECG measuring device).

The ECG electrode may have a wireless interface to transfer data from or to the ECG electrode. Transmission of an ECG measurement signal may take place via this interface from the integrated ECG measuring device to a more remote signal receiving device that, for example, is arranged on another processing unit that then processes the ECG measurement signal and, for example, is configured to trigger an imaging device and/or to display the ECG measurement signal on a screen and/or to save or record the ECG measurement signal.

To obtain the least possible electromagnetic interference signal influence possible, the contact element carrier may have electromagnetic shielding for the first skin contact element and/or the second skin contact element and/or the first connection element and/or the second connection element and/or the ECG measuring device. In one embodiment, all these components are correspondingly well shielded. Likewise, insofar as electric supply lines are used, these may also be correspondingly shielded. The skin contact elements are not shielded in the direction of the skin, as the best possible contact with the skin is desired.

To obtain particularly good contact, the first skin contact element and/or the second skin contact element at least partially exhibit a contact surface with an electrically conductive plastic for contact with the skin (e.g., in the manner of a dry electrode). In other words, at least the contact surface of the respective skin contact elements is, for example, formed by the plastic, or a skin contact element made entirely of such conductive plastic is used.

In one embodiment, the first skin contact element and/or the second skin contact element at least partially exhibit a contact surface with an uneven surface structure, as this may also lead to the improvement of the contact of the skin.

As aforementioned, besides the ECG electrode according to one or more of the present embodiments, a further ECG electrode for measuring or tapping a third potential is used in an ECG electrode set. In one embodiment, one of these electrodes is configured as a head electrode. In one embodiment, this is a face electrode or an ear electrode (e.g., “ear ECG electrode”). For example, an ear electrode may be configured as an ear clamping electrode that is clamped to the earlobe.

There are already ear clamps that are used as pulse oximeter sensors. These ear clamps have LEDs and a photosensor for fluoroscopy and for measurement of the light intensity in order to measure the pulse in this way. Likewise, ear electrodes for deriving EEG signals are known. Within the scope of the method according to one or more of the present embodiments, such an electrode is now to be used on the head (e.g., an ear ECG electrode of the aforementioned type) in order to detect a third potential of the patient. When measuring an ECG measurement signal by deriving the potential, for example, on the left arm and on the left ear, an excellent Einthoven I derivative may be provided. In one embodiment, either the first potential or the second potential, both of which are tapped with the ECG electrode according to one or more of the present embodiments for encompassing the extremity (e.g., the clamping electrode on the arm or wrist of the patient) may then be used as a reference potential to replace the neutral electrode.

In this respect, the use of a head electrode (e.g., a face or ear ECG electrode of the aforementioned type) for detecting a potential for an ECG measurement signal is also independently advantageous even if, for example, two conventional wrist clamps or similar electrodes are used, as it may then also be unnecessary to attach a further electrode to one of the legs of the patient. The head or the ear of the patient are considerably easier for the operating personnel of an imaging device to access than the leg, and the cabling of the electrodes among each other and to an ECG measuring device is considerably less disturbing for the patient than the use of a leg electrode.

### BRIEF DESCRIPTION OF THE DRAWINGS

In the various figures, same components have same reference numerals. The figures are generally not to scale.

FIG. 1 is a diagrammatic view of a conventional clamp ECG electrode according to the prior art for detecting a potential on the arm or leg of a patient;

FIG. 2 is a diagrammatic view of the use of three conventional clamp ECG electrodes according to FIG. 1 for deriving an ECG derivative on a human patient, where a conventional ECG clamping electrode is fitted on the left arm, the right arm, and on the right leg, respectively;

FIG. 3 is a diagrammatic view of a first exemplary embodiment of an ECG electrode for detecting two different potentials on an arm or leg of a patient;

FIG. 4 is a diagrammatic view of an exemplary embodiment of an ear ECG electrode according to an embodiment for detecting a potential on an ear of a patient;

FIG. 5 is a diagrammatic view of the use according to an embodiment of an ECG electrode according to FIG. 3 on the left arm or wrist and an ECG electrode according to FIG. 4 on the left ear for deriving an ECG derivative;

FIG. 6 is a diagrammatic view of a second exemplary embodiment of an ECG electrode similar to the ECG electrode in FIG. 3, but with an integrated ECG measuring device; and

FIG. 7 is a diagrammatic view of an exemplary embodiment of a skin contact element with a structured contact surface.

### DETAILED DESCRIPTION

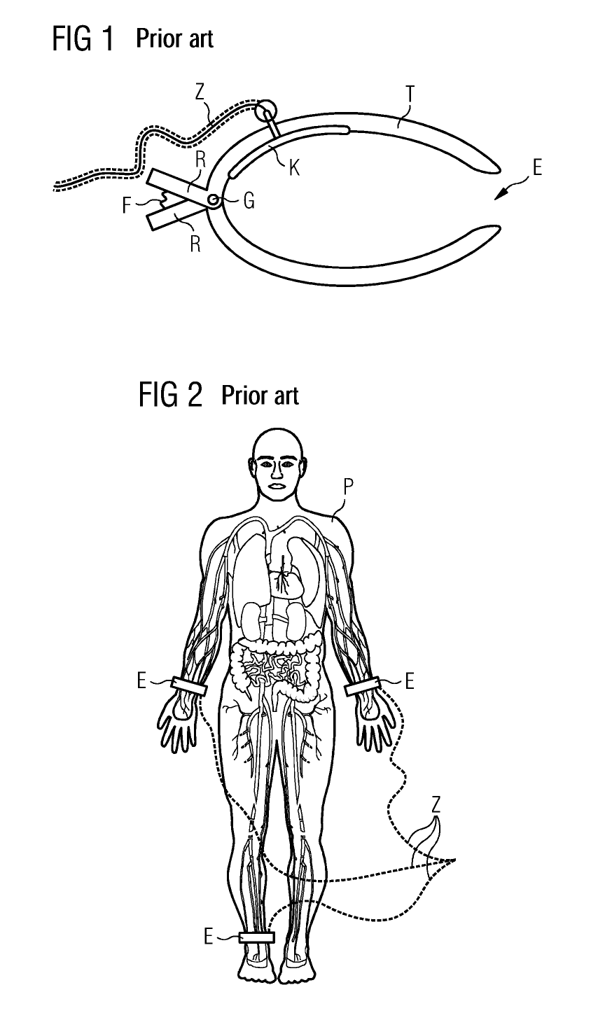

The conventional prior art for measuring a simple electrocardiography (ECG) measurement signal according to an Einthoven I derivative is shown briefly with reference to FIGS. 1 and 2. For this purpose, for example, three wrist or ankle clamps (e.g., “clamping electrodes” or “ECG clamping electrodes”) are used, as shown diagrammatically in FIG. 1. Such clamping electrodes E have a contact element carrier T that, for example, may be constructed in two parts from two flattened ring segments (e.g., of insulating plastic) that are movably coupled to one another via a hinge G. The individual ring segments on the hinge G each have grip sections R that are pushed apart by a spring F (represented symbolically). To apply the ECG clamping electrodes E, these grip elements R may be pressed together against the spring force F. As a result of this, the ECG clamping electrodes E on the side facing away from the hinge G open further and may thus be pushed over the wrist or the forearm. Instead of the spring, the hinges may also be configured to be suitably resilient. If the grip sections R are released again, the two ring segments are pressed against the skin of the patient P, and a skin contact element K (e.g., a “contact”) arranged on one of the ring segments on the inner side may be pressed against the skin of the patient P. On this skin contact element K, which is a metal element, there is a connection element on the back (e.g., in the form of a plug socket), into which a plug of a supply cable or a supply line Z may be inserted. On the other side, this supply cable may be inserted into a socket of an input channel of an ECG measuring device to reduce the potential on the skin of the patient at the point at which the skin contact element K presses against the skin of the patient. The entire structure of such an ECG clamping electrode E is known to a person skilled in the art, and therefore, does not need to be explained in more detail.

FIG. 2 shows the use of three such conventional ECG clamping electrodes E for measuring an Einthoven I derivative. On the patient, corresponding ECG clamping electrodes E are attached to the wrists or slightly above on both arms, and a third ECG clamping electrode E is attached to the left leg (e.g., the three supply lines Z then lead to a conventional ECG measuring device not shown in FIG. 2). A potential difference for obtaining ECG measurement signals between the two ECG clamping electrodes E on the wrists is measured. The potential measurement with the ECG clamping electrode E on the leg is used as a reference potential (e.g., this is the neutral electrode).

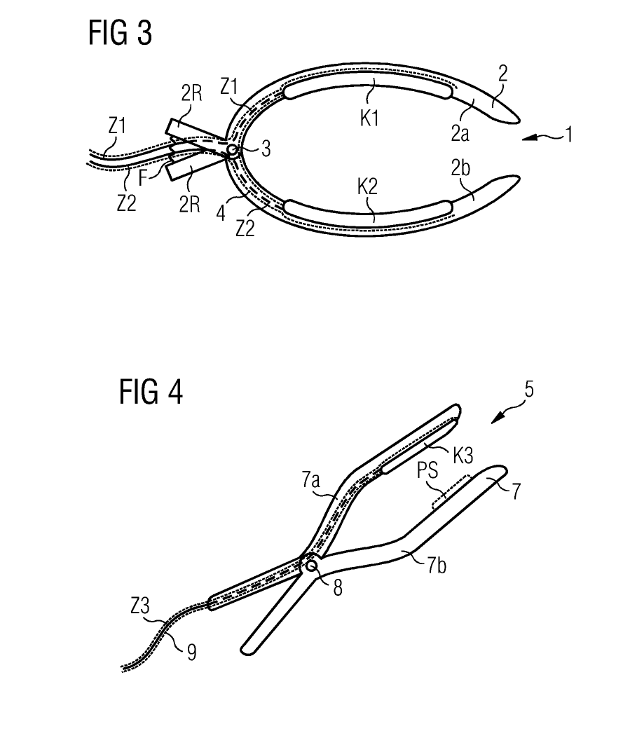

FIG. 3 shows an exemplary embodiment of an ECG electrode 1 (e.g., as an ECG clamping electrode 1 or wrist or ankle clamp). The contact element carrier 2 is made of plastic and may be shaped or configured in the same way as the ECG clamping electrodes according to the prior art, as explained with reference to FIG. 1. This provides that the contact element carrier 2 has two flattened ring segments 2a, 2b of plastic that are coupled to one another via a hinge 3. Each of the two flattened ring segments 2a, 2b has a grip section 2R on the other side of the hinge 3. The grip sections 2R may be pressed together against the spring force of a spring F or by the resilient design of the hinge to open and fasten the ECG clamping electrode 2 on an extremity A (e.g., an arm A) of the patient P (see FIG. 5). Unlike the ECG clamping provided by the electrodes according to the prior art, according to FIG. 1, however, there is now a skin contact element K1, K2 on the inside on each of the two ring segments 2a, 2b. Both skin contact elements K1, K2 are connected with connection elements Z1, Z2 that, for example, are routed in a common two-core cable to an ECG measuring device and are there connected to various input channels. The ECG clamping electrode 2 may have two connection elements in the form of two plug contacts such that a connection via two cables may be possible. However, a combination with a two-core cable may be provided; a common plug contact may be used, for example, in the form of a coaxial plug or the like. With the aid of this ECG electrode 1, two potentials may be tapped simultaneously on the wrist or forearm of the patient P or also on the leg or ankle (e.g., a first potential that is used for a differential measurement with regard to a third potential on another part of the body of the patient, and a reference potential that replaces the neutral electrode).

The skin contact elements K1, K2 and the connection elements Z1, Z2 are shielded as well as possible by a shield 4 only shown diagrammatically.

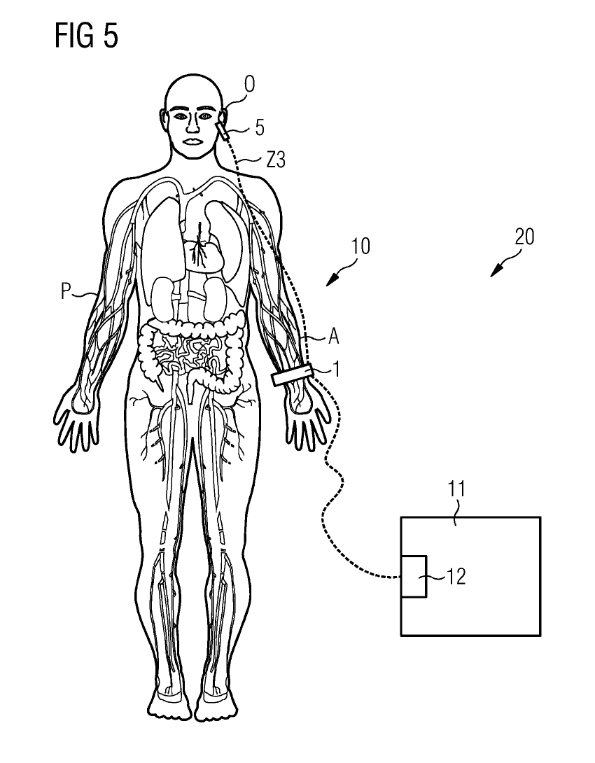

FIG. 4 shows a further embodiment of an ECG electrode 5 that may be used as a further ECG electrode 5 inside an ECG electrode set 10. This is likewise a type of ECG clamping electrode, but now as an ear ECG electrode 5 that may be clipped to an earlobe of a patient. With this ear ECG electrode 5, the contact element carrier 7 may also include two plastic parts 7a, 7b that are connected to one another via a hinge 8. The two plastic parts 7a, 7b may be opened with respect to one another against a spring force and may be pressed together again by the spring force such that the ECG electrode 5 may be very easily clamped to an earlobe of the patient.

On the inner side of one of the two parts 7a, 7b of the contact element carrier 7, there is a further skin contact element K3 for tapping a potential on the earlobe of the patient P. This skin contact element K3 has a connection element Z3 (e.g., also a cable). As in the case of the ECG clamping electrodes 1 according to FIG. 3, the skin contact element K3 and the connection element Z3 are shielded as well as possible by a shield 9.

In addition, the ear ECG electrode 5 may optionally have another pulse oximeter sensor PS that is only shown diagrammatically. Usually this has at least one LED and one device for measuring the LED light that passes through the earlobe. This provides that this pulse oximeter sensor PS has several components that may be found on the various parts 7a, 7b of the contact element carrier 7. The signal measured with the pulse oximeter sensor PS may then, for example, also be transmitted via the cable for the connection element Z3 if a multicore cable is used.

FIG. 5 shows an ECG measuring system 20 with such an electrode set 10 including at least one ECG clamping electrode 1, according to FIG. 3, and one ear ECG electrode 5, according to FIG. 4, in the application on a patient P. As shown, the ear ECG electrode 5 is connected to the left ear 0 of the patient P, and the potential measured on the skin contact element K3 is first forwarded via the connection element Z3 or supply cable Z3 in the direction of an ECG clamping electrode 1 according to one or more of the present embodiments, as shown in FIG. 3. This ECG clamping electrode 1 is arranged as a wrist clamp on the left arm A of the patient P.

From there, the supply line, together with the supply lines of the skin contact elements K1, K2 of the ECG clamping electrode 1, is then forwarded to an input interface 12 of an ECG measuring device 11. The supply lines from the O ear ECG electrode 5 and the ECG clamping electrode 1 may be routed in parallel from the left wrist, if necessary, even inside a multicore cable. The input interface 12 of the ECG measuring device 11 may then be configured accordingly. In doing so, for example, a differential measurement is performed between the third skin contact element K2 on the ear 0 and the first skin contact element K1 on the ECG clamping electrode 1 on the wrist. The second skin contact element K2 on the ECG clamping electrode 1 is, for example, used as a replacement for the neutral electrode (e.g., as a Right Leg Drive) to obtain the reference potential.

As shown, there is only one first supply line or cable connection for the connection of the ear ECG electrode 5 between the left ear and the left arm and a second supply line or cable connection that leads to the ECG measuring device 11. Advantageously, no cables of any kind are therefore routed over the body of the patient, which is advantageous for imaging. In addition, both the ECG clamping electrode 1 according to FIG. 3 and the ear ECG electrode 5 according to FIG. 4 may be attached by the personnel to the patient P very quickly without the latter needing to be undressed and without assistance being needed. In practice, an excellent ECG measurement signal, similar to an Einthoven derivative I, may be measured on the left ear and on the left wrist via the derivative. The patient P is only very slightly restricted in freedom of movement in the process.

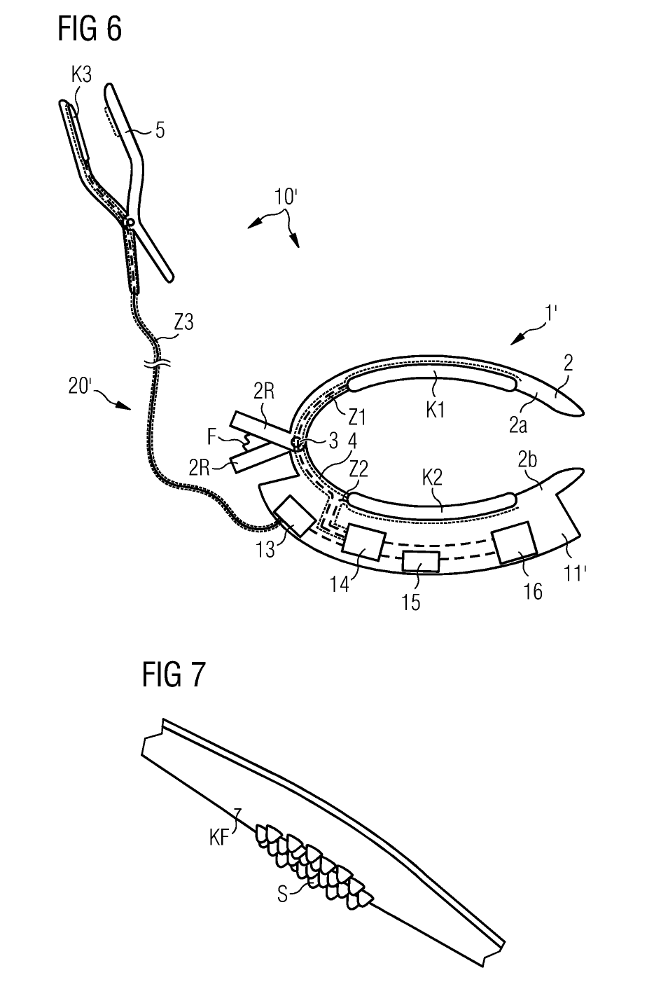

FIG. 6 shows a further exemplary embodiment of an ECG measuring system 20′ with an electrode set 10′, with an ECG clamping electrode 1′ according to one or more of the present embodiments in the manner of a wrist clamp, similar to the one shown in FIG. 3. Unlike the exemplary embodiment according to FIG. 3, however, an ECG measuring device 11′ is included on the outside of the contact element carrier 2 (e.g., the ECG measuring device 11′ is integrated into the ECG clamping electrode 1′). For example, a housing (e.g., of plastic) may also be mounted on one of the two plastic ring segments 2a, 2b (e.g., including a processing unit 14 (a processor) to process the signals or potentials that are detected by the skin contact elements K1, K2). To measure the third, more remote potential, an ear ECG electrode 5 that is connected to an input interface 13 of the integrated ECG measuring device 11′ via a supply line Z3 may be used. The potential measured on the ear ECG electrode 5 and the potentials measured by the first skin contact element K1 and second skin contact element K2 are used to derive an ECG measurement signal in the processing unit 14. This signal may then be transmitted from the processing unit 14 to a wireless interface 15 that transmits the signal to a further processing unit (not shown) in which the ECG measurement signal is further used, for example, to trigger an imaging device and/or to show and/or store the ECG measurement signal.

For the supply of energy, there is an energy storage system 16 in the integrated ECG measuring device 11, for example, in the form of a replaceable accumulator 16 or batteries. Likewise, a charging system for charging the accumulator may be used (e.g., a wireless charging station that charges the accumulator when the ECG clamping electrode 1′ is brought into such a charging station). For example, the processing unit 14 and the wireless interface 15 are supplied with the required energy by this energy storage system 16.

This integrated ECG measuring device 11′ may also be well shielded from electromagnetic interference. When using such an ECG measuring system 20′ with an integrated ECG measuring device 11′ in the ECG electrodes 1′, the patient P has even greater freedom of movement, as there is only a single cable connection between the ear ECG electrode 5 and the ECG clamping electrode 1′ on the wrist.

To facilitate the contact of the skin of the patient P with the skin contact elements K1, K2, K3, the skin contact elements K1, K2, K3 may have a contact surface KF with a conductive plastic coating or are made entirely of conductive plastic. At least part of the contact surface KF has an uneven surface structure S that improves contact still further. Suitable conductive plastics are, for example, acrylonitrile butadiene styrene (ABS) or ethylene propylene rubber (EPDM), which may be displaced by appropriate additives such as, for example, carbon or carbon black.

As shown by the aforementioned exemplary embodiments, one or more of the present embodiments offer good to very good properties with regard to all the aforementioned parameters (e.g., with regard to applicability and patient-friendliness). The impact on the patient is therefore only minimal; the patient need not undress, and the patient retains freedom of movement. As a result, the ECG electrodes may even be applied to the patient in the waiting room before examination, and data may already recorded there. Application is very easy. While special points are to be identified on the body for adhesive or suction electrodes, the left arm and the left ear are clearly and easily identifiable. For example, the one or more of the present embodiments are very well suited to an application in the computer tomograph, as there is no metal in the relevant scan regions.

The aforementioned devices and methods described in detail are only exemplary embodiments that may be modified in a variety of ways by a person skilled in the art without departing from the scope of the invention. For example, the wrist electrodes may also be configured as bands or the like instead of as clamp electrodes. The use of the indefinite article “a” or “an” does not rule out the possibility of the features concerned also being present multiple times. The terms “unit” or “element” do not rule out the component concerned consisting of several interacting sub-components that may possibly also be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An electrocardiography (ECG) electrode comprising:
a contact element carrier for encompassing a first extremity of a patient;
a first skin contact element arranged on the contact element carrier with a first connection element for detecting a first potential on the first extremity; and
at least one second skin contact element arranged on the contact element carrier with a second connection element for detecting a second potential, the second potential being different than the first potential on the first extremity.

2. The ECG electrode of claim 1, wherein the contact element carrier has a band element or a ring element, and the first skin contact element and the at least one second skin contact element are arranged on an inner side of the contact element carrier pointing towards skin of the patient in essentially opposite sections of the contact element carrier when the ECG electrode is used with the patient.

3. The ECG electrode of claim 1, wherein the contact element carrier, for encompassing a forearm or lower leg, is configured as a wrist clamp, an ankle clamp, or a combination thereof or as a wrist band, an ankle band, or a combination thereof.

4. The ECG electrode of claim 1, wherein the first skin contact element, the at least one second skin contact element, or the first skin contact element and the at least one second skin contact element each has a contact surface with the first extremity that is at least 0.5 cm², is 5 cm² maximum, or a combination thereof.

5. The ECG electrode of claim 1, wherein the contact surface with the first extremity, respectively, is at least 1 cm², 3 cm² maximum, or a combination thereof.

6. The ECG electrode of claim 1, wherein the contact surface with the first extremity, respectively, is at least 1.5 cm², 2.5 cm² maximum, or a combination thereof.

7. The ECG electrode of claim 1, further comprising an integrated ECG measuring device.

8. The ECG electrode of claim 7, further comprising an energy storage system configured to operate the ECG electrode, a wireless interface configured to transfer data from or to the ECG electrode, or the energy storage system and the wireless interface.

9. The ECG electrode of claim 8, wherein the wireless interface is configured to transmit an ECG measurement signal from the integrated ECG measuring device to a signal receiving device.

10. The ECG electrode of claim 1, wherein the contact element carrier has a shield for the first skin contact element, the at least one second skin contact element, the first connection element, the second connection element, the integrated ECG measuring device, or any combination thereof.

11. The ECG electrode of claim 1, wherein the first skin contact element, the at least one second skin contact element, or the first skin contact element and the at least one second skin contact element at least partially comprises a contact surface with an electrically conductive plastic;
wherein the first skin contact element, the at least one second skin contact element, or the first skin contact element and the at least one second skin contact element at least partially exhibit a contact surface with an uneven surface structure; or
a combination thereof.

12. An electrocardiography (ECG) electrode set comprising:
a number of ECG electrodes, wherein at least one of the ECG electrodes comprises:
a contact element carrier for encompassing a first extremity of a patient; and
a first skin contact element arranged on the contact element carrier with a first connection element for detecting a first potential on the first extremity; and
at least one second skin contact element arranged on the contact element carrier with a second connection element for detecting a second potential, the second potential being different than the first potential on the first extremity.

13. The ECG electrode set of claim 12, wherein one of the ECG electrodes is configured as a head electrode with an integrated pulse oximeter sensor.

14. The ECG electrode set of claim 13, wherein the one ECG electrode is configured as a face or ear electrode.

15. An electrocardiography (ECG) measuring system comprising:
an ECG electrode set comprising:
a number of ECG electrodes, wherein at least one of the ECG electrodes comprises:
a contact element carrier for encompassing a first extremity of a patient; and
a first skin contact element arranged on the contact element carrier with a first connection element for detecting a first potential on the first extremity; and
at least one second skin contact element arranged on the contact element carrier with a second connection element for detecting a second potential, the second potential being different than the first potential on the first extremity; and
an ECG measuring device.

16. A method for detecting an electrocardiography (ECG) measurement signal, the method comprising:
tapping, on a first extremity of a patient, by an ECG electrode, a first potential and a second potential different than the first potential; and
detecting at least a third potential on a further part of the body of the patient.

17. The method of claim 16, wherein the first potential or the second potential is a reference potential.

18. The method of claim 16, wherein the third potential is detected on a further extremity or the head of the patient.

19. The method of claim 18, wherein the third potential is detected on an ear of the patient.

20. A method for using a head electrode, the head electrode including a contact element carrier for encompassing a first extremity of a patient, a first skin contact element arranged on the contact element carrier with a first connection element for detecting a first potential on the first extremity, and at least one second skin contact element arranged on the contact element carrier with a second connection element for detecting a second potential, the second potential being different than the first potential on the first extremity, the method comprising:
detecting a potential for an electrocardiography (ECG) measurement signal using the head electrode.
